(19) United States
(12) Patent Application Publication
(10) Pub. No.: US 2024/0398586 A1
(43) Pub. Date: Dec. 5, 2024

(54) Glenoid Trial Implant for Reverse Total Shoulder Prosthesis

(71) Applicant: FX Shoulder Solutions, Viriat (FR)

(72) Inventor: Jean-Marie Daudet, Enbresse (FR)

(73) Assignee: FX Shoulder Solutions, Viriat (FR)

(21) Appl. No.: 18/755,854

(22) Filed: Jun. 27, 2024

Related U.S. Application Data
(63) Continuation of application No. 17/653,892, filed on Mar. 8, 2022.

(30) Foreign Application Priority Data
Mar. 12, 2021 (FR) FR2102495

(51) Int. Cl. A61F 2/46; A61F 2/30; A61F 2/40

(52) U.S. Cl. CPC A61F 2/4684; A61F 2/30749; A61F 2002/30616; A61F 2002/4085

(57) ABSTRACT

The invention relates to a glenoid trial implant for reverse total shoulder prosthesis including a metaglene intended to be assembled in a stabilized and temporary manner inside the glenoid fossa of a patient who will receive surgery, and a glenosphere integrally assembled to the metaglene.

### CROSS REFERENCE TO RELATED APPLICATION

This application is a continuation of application Ser. No. 17/653,892 filed Mar. 8, 2022, entitled, Glenoid Trial Implant For Reverse Total Shoulder Prosthesis, pending, claimed priority over French Patent Application FR2102495 filed Mar. 12, 2021, the content of which is incorporated herein by reference.

### FIELD OF THE INVENTION

The invention relates to a glenoid trial implant for reverse shoulder prosthesis, intended to be temporarily attached to the patient's glenoid fossa.

### BACKGROUND OF THE INVENTION

A reverse total shoulder prosthesis comprises a humeral implant, in other words a part attached in the patient's humerus, and a glenoid implant, in other words a part attached on the patient's glenoid fossa (also called glenoid cavity). These two implants are hinged to each other to restore the mobility of the arm of the patient receiving surgery.

In case of a “reverse” total shoulder prosthesis, the center of rotation of the joint is shifted to the glenoid implant, unlike an anatomic prosthesis where it remains on the humeral implant.

This modification of the position of the center of rotation of the joint generates a modification of the muscles solicited to move the arm. In the present case, the deltoid muscle is solicited while, for an anatomic prosthesis, the rotator cuff muscles are solicited, i.e. the subscapularis, supraspinatus, infraspinatus and teres minor in addition to the deltoid. This modification of the muscles involved can be explained by irreparable damage of all or some of the rotator cuff muscles, such damage possibly occurring in old persons, in particular.

The reverse shoulder prosthesis therefore involves soliciting the deltoid muscle which is anchored to the humerus in a more distal manner than the rotator cuff muscles. Consequently, for the deltoid muscle to operate during the first degrees of abduction, the center of rotation of the prosthesis must be shifted to the glenoid fossa. In addition, the deltoid muscle must be tensed so that it can be used for the internal and external rotations. This tension of the deltoid muscle can be achieved by lateralizing and/or longitudinally extending the humerus. In other words, the humerus is lateralized and/or lowered relative to its natural position in order to increase the tensions applied to the deltoid muscle.

This lateral and/or longitudinal shift of the center of rotation thus located on the glenoid implant can be obtained respectively by increasing the total thickness of the implant (variability of the glenosphere diameter or of the metaglene thickness) or by creating an offset between the glenosphere and the metaglene forming the glenoid implant (glenoid implants currently exist for which the glenosphere is offset relative to the metaglene in order to lower the center of rotation more or less depending on the glenosphere diameter).

Consequently, the choice of glenoid implant is a determining factor for the success of the surgery and the lifetime of the total shoulder prosthesis. Since these implants are sterile components under vacuum which cannot be sterilized again, it is important to know which implant will be used before opening the packaging containing the implant, to avoid desterilizing an implant that would be unsuitable for the patient, and thus make it unusable.

Before choosing the glenoid implant most suitable for the patient, without however removing it from its sterile packaging, it is known to use software allowing 3D planning during preoperative periods. However, these solutions have several disadvantages. They require sophisticated and expensive electronic equipment. In addition, they involve extensive preoperative work by the surgeon, which means that he/she is not available for other tasks during this period. Lastly, they cannot guarantee that the most suitable implant will be chosen since the planning software does not take into account all the variables involved in the joint between the humerus and the glenoid fossa, for example the ligament tension resulting from the chosen combination of the humeral and glenoid implants and their positioning.

### OBJECT AND SUMMARY OF THE INVENTION

The invention aims in particular to guarantee that the glenoid implant most suitable for the patient will be chosen, in order to reduce the number of unnecessarily desterilized implants. The invention also aims to significantly shorten the preoperative time required by the surgeon. Lastly, the invention aims to avoid costly solutions such as those requiring the use of planning software, by proposing an inexpensive solution.

Thus, the invention relates to a glenoid trial implant for reverse total shoulder prosthesis comprising:
a metaglene having an axis of revolution and being intended to be assembled in a stabilized and temporary manner inside the glenoid fossa of a patient who will receive surgery, and
a glenosphere having an axis of revolution and being integrally assembled to the metaglene and intended to be received inside a cupula of a humeral trial implant.

A glenoid trial implant corresponds to an implant temporarily attached by the surgeon during the operative period to perform trials in order to determine the final position of the center of rotation of the shoulder joint. For reverse shoulder prostheses, the center of rotation of the shoulder joint corresponds to the axis of revolution of the glenosphere. The trial implant will be removed, either so that another trial implant can be tested by the surgeon or, when the final position of the center of rotation of the shoulder joint has been determined, to be replaced by the final corresponding implant.

The metaglene of the trial implant according to the invention can be circular, ovoid or pear-shaped.

The glenosphere of the trial implant according to the invention can be hemispherical or be a glenosphere of more than a half-sphere.

Thus, the surgeon can determine in real-time the possible shoulder mobility using the combination of a glenoid trial implant according to the invention and a humeral trial implant. The surgeon can in fact test the ligament and muscular tension resulting from the chosen combination installed, allowing him/her to define the glenoid implant suitable for the operation in progress with certainty and easily since there is no need for planning or navigation software.

Consequently, the final glenoid implant suitable for the operation in progress can be removed from its sterile packaging only when the surgeon is certain that it is the right implant. The invention therefore significantly reduces the risk of unnecessarily opening the packaging of final glenoid implants which would subsequently prove to be unsuitable for the operation in progress.

According to other optional characteristics of the invention taken alone or in combination:
the axes of revolution of the metaglene and of the glenosphere coincide;
the axes of revolution of the metaglene and of the glenosphere intersect;
the axes of revolution of the metaglene and of the glenosphere are strictly parallel;
the axes of revolution of the metaglene and of the glenosphere are separated from each other by a distance of between 0.5 and 5 mm, preferably between 1 and 3 mm;
the metaglene comprises a stop designed to act as a spacer between the glenosphere and the glenoid fossa when the trial implant is assembled inside the glenoid fossa, the thickness of said stop being less than or equal to 10 mm, preferably less than or equal to 6 mm;
the metaglene and the glenosphere are made in one piece, forming a monoblock implant;
the glenoid trial implant comprises means for assembling the metaglene and the glenosphere;
The metaglene and/or the glenosphere are made from plastic polymer or stainless steel or titanium and an alloy of these materials.

The invention also relates to a set comprising several glenoid trial implants according to any one of the possible variants.

Thus, the surgeon can be provided with a set (possibly consisting of a kit or a box of trial implants) comprising a plurality of glenoid trial implants according to the invention. This plurality of glenoid trial implants allows the surgeon to choose from all the possible combinations required to best adjust, during the operation, the center of rotation of the shoulder joint according to the patient's morphological characteristics.

The invention also relates to a method for attaching the reverse total shoulder prosthesis in a patient, comprising the steps of:
a) attaching a glenoid trial implant according to any one of claims 1 to 9, in the patient's glenoid fossa,
b) attaching a humeral trial implant in the patient's humerus,
c) checking that the glenoid and humeral trial implants are compatible in order to position the center of rotation of the shoulder joint at the right location according to the patient's morphological characteristics.

According to other optional characteristics of the method according to the invention taken alone or in combination:
if the glenoid and humeral trial implants are found to be incompatible, at least one of steps a), b) or c) is repeated, with a glenoid trial implant and/or a humeral trial implant of different dimensions;

if the glenoid and humeral trial implants are found to be compatible, they are removed and replaced by final glenoid and humeral implants of identical dimensions.

### BRIEF DESCRIPTION OF THE FIGURES

The invention will be better understood on reading the following description, given solely by way of example and with reference to the accompanying drawings in which:

FIG. 1 is a set of diagrammatic views showing the humoral and glenoid implants forming a reverse total shoulder prosthesis;

FIG. 2 is a set of diagrammatic views showing glenoid implants according to several variants of the invention;

FIG. 3 is a set of diagrammatic views showing glenoid implants according to several variants of the invention;

FIG. 4 is a set of diagrammatic views showing glenoid implants according to several variants of the invention.

### DETAILED DESCRIPTION

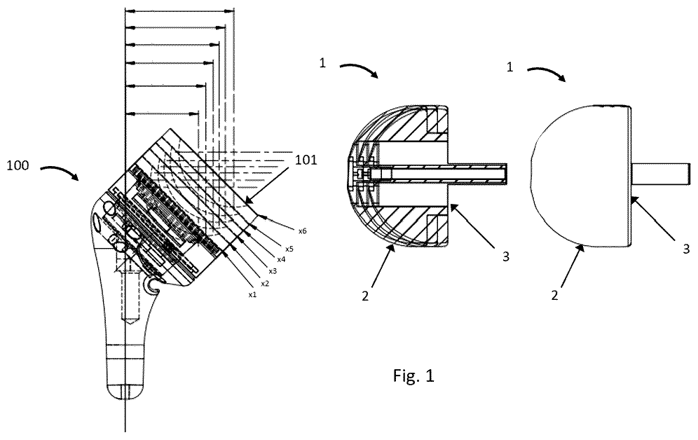

FIGS. 1 to 4 show glenoid trial implants for reverse total shoulder prosthesis, designated by the general reference 1.

As shown on FIG. 1, the glenoid trial implant 1 according to the invention comprises a metaglene 3 having an axis of revolution d1 and being intended to be assembled in a stabilized and temporary manner inside the glenoid fossa (not shown) of a patient who will receive surgery. In the variants shown on the figures, the metaglene 3 is circular. The implant 1 also comprises a glenosphere 2 having an axis of revolution (d2) and being integrally assembled to the metaglene 3 and intended to be received inside a cupula 101 of a humeral trial implant 100. In the variants shown on the figures, the glenosphere 2 is more than a half-sphere.

Thus, the surgeon has a trial implant 1 that he/she can attach to the glenoid fossa in order to define, during the operation, the best position for the center of rotation of the shoulder joint. When such a position is reached, the surgeon can choose the final glenoid implant whose shape corresponds to the trial implant 1. Thus, the final implant is removed from its sterile packaging only when the surgeon is certain that it is the right implant to be used.

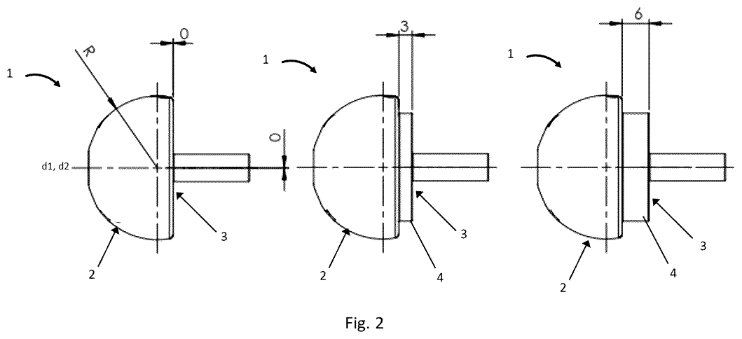

According to alternative embodiments of the invention shown on FIG. 2, the axes of revolution d1 and d2 of the metaglene 3 and of the glenosphere 2 coincide, which means that these two elements can be centered relative to each other. This configuration is particularly advantageous when there is no need to longitudinally lower the center of rotation of the shoulder joint (which corresponds to the axis of revolution d2 of the glenosphere 2).

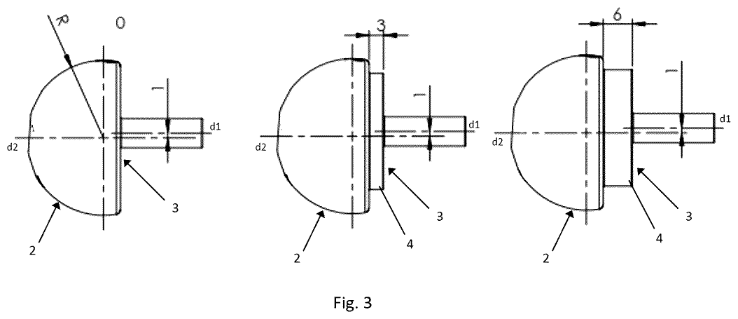

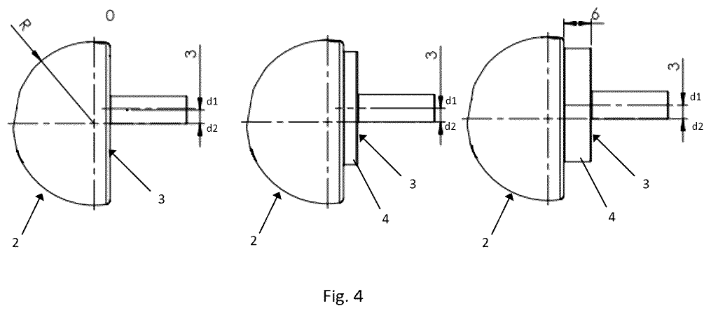

Alternatively, as shown on FIGS. 3 and 4, the axes of revolution d1 and d2 of the metaglene 3 and of the glenosphere 2 are strictly parallel, which means that these two elements are offset relative to each other. According to the variants of the invention shown on FIG. 3, the metaglene 3 and the glenosphere 2 are longitudinally offset relative to each other, with respect to the patient's body, by a distance of 1 mm, thereby finally lowering the position of the center of rotation of the shoulder joint, which also lowers the humerus longitudinally. This tenses the deltoid muscle in a longitudinal direction. This offset also allows the metaglene to be positioned slightly higher or lower according to the patient's morphological characteristics. Such a variation also allows the center of rotation of the shoulder joint to be positioned in the most suitable location. According to the variants of the invention shown on FIG. 4, the axes of revolution d1 and d2 are separated by a distance of 3 mm, which means that the center of rotation of the joint can be lowered even further and therefore, finally, that a greater tension can be applied on the deltoid muscle.

The metaglene 3 comprises a stop 4 (visible on FIGS. 2 to 4) designed to act as a spacer between the glenosphere 2 and the glenoid fossa when the trial implant 1 is assembled inside the latter. As shown, the thickness of this stop 4 varies up to 6 mm. This variation in the thickness of the stop 4 allows lateralization of the center of rotation of the shoulder joint and, consequently, lateralization of the humerus once the shoulder prosthesis has been assembled. As a result, a greater tension is applied on the deltoid muscle in the lateral direction.

The deltoid muscle can therefore be tensed sufficiently so that it can operate from the first degrees of abduction as a replacement for the muscles forming the rotator cuff.

Lastly, the diameter of the glenosphere 2 of a glenoid trial implant 1 can also be varied to further offset its axis of revolution d2 (and therefore vary the center of rotation of the shoulder joint longitudinally and/or laterally). The diameter of such glenospheres 2 can vary between 30 and 44 mm.

The glenoid trial implant 1 can be monoblock, which means that the metaglene 3 and the glenosphere 2 are made in one piece and cannot be separated from each other.

Alternatively, the implant 1 comprises assembly means used to integrally attach the metaglene 3 and the glenosphere 2 so that they do not separate when the implant 1 is attached inside the glenoid fossa or when the surgeon performs tests during the operation. Such assembly means are known to those skilled in the art. As non-limiting examples, clips or a screw/nut combination, etc. can be used.

The surgeon can be provided with a set of several glenoid trial implants 1 according to different variants of the invention, in the form of a kit. The surgeon can therefore choose from all the possible variants of glenoid trial implants 1 required to best adjust the center of rotation of the shoulder joint according to the patient's morphological characteristics. Once the center of rotation of the shoulder joint has been placed in the right position, the surgeon only has to choose the final glenoid implant of shape identical to that of the glenoid trial implant 1 used to reach said position.

The invention is not limited to the embodiments described and other embodiments will be clearly apparent to those skilled in the art. In particular, glenospheres of diameters different from those mentioned can be used, or the metaglene can be further offset relative to the glenosphere.

### LIST OF REFERENCES

1: glenoid trial implant
2: glenosphere
3: metaglene
4: stop
100: humoral trial implant
101: cupula
d1: axis of revolution of the metaglene
d2: axis of revolution of the glenosphere

1-11. (canceled)

12. A method for attaching a reverse total shoulder prosthesis in a patient, comprising the steps of:
a) attaching a glenoid trail implant in a patient's glenoid fossa, wherein the glenoid trail implant comprises: a metaglene having an axis of revolution and adapted to be temporarily assembled inside a glenoid fossa of a patient having surgery, and a glenosphere having an axis of revolution and being integrally assembled to the metaglene and intended to be received inside a cupula of a humeral trial implant,
b) attaching a humeral trial implant in the patient's humerus,
c) checking that the glenoid and humeral trial implants are compatible in order to position the center of rotation of the shoulder joint at the right location according to the patient's morphological characteristics.

13. The method for attaching a reverse total shoulder prosthesis in a patient, according to claim 12, wherein if the glenoid and humeral trail implants are found to be incompatible, at least one of steps a), b) or c) is repeated, with at least one of a glenoid trial implant and a humeral trial implant of different dimensions.

14. The method for attaching a reverse total shoulder prosthesis in a patient, according to claim 12, wherein if the glenoid and humeral trail implants are found to be compatible, they are removed and replaced by final glenoid and humeral implants of identical dimensions to the glenoid trail implant and humeral trail implant, respectively.

15. The method for attaching a reverse total shoulder prosthesis in a patient, according to claim 12, wherein the axis of revolution of the metaglene and the glenosphere coincide.

16. The method for attaching a reverse total shoulder prosthesis in a patient, according to claim 12, wherein the axis of revolution of the metaglene and the axis of revolution of the glenosphere intersect with each other.

17. The method for attaching a reverse total shoulder prosthesis in a patient, according to claim 12, wherein the axis of revolution of the metaglene and the glenosphere are parallel.

18. The method for attaching a reverse total shoulder prosthesis in a patient, according to claim 17, wherein the axis of revolution of the metaglene and the glenosphere are separated from each other by a distance of between 0.5 and 5 mm.

19. The method for attaching a reverse total shoulder prosthesis in a patient, according to claim 12, wherein the metaglene comprises a stop designed to act as a spacer between the glenosphere and the glenoid fossa when the trial implant is assembled inside the glenoid fossa, the stop having a thickness of less than or equal to 10 mm.

20. The method for attaching a reverse total shoulder prosthesis in a patient, according to claim 12, wherein the metaglene and the glenosphere are made in one piece, forming a monoblock implant.

21. The method for attaching a reverse total shoulder prosthesis in a patient, according to claim 12, wherein the glenoid trial implant comprises an assembling device to assemble the metaglene and the glenosphere.

22. The method for attaching a reverse total shoulder prosthesis in a patient, according to claim 12, wherein the metaglene and/or the glenosphere are made from plastic polymer, stainless steel, titanium, or an alloy of these materials.

23. The method for attaching a reverse total shoulder prosthesis in a patient, according to claim 12, wherein a set comprising several glenoid trial implants with different dimensions.

24. The method for attaching a reverse total shoulder prosthesis in a patient, according to claim 13, wherein if the glenoid and humeral trail implants are found to be compatible, they are removed and replaced by final glenoid and humeral implants of identical dimensions to the glenoid trail implant and humeral trail implant, respectively.
